(19) United States
(12) Patent Application Publication
(10) Pub. No.: US 2023/0233564 A1
(43) Pub. Date: Jul. 27, 2023

(54) METHODS FOR BREAST CANCER TREATMENT AND PREDICTION OF THERAPEUTIC RESPONSE

(71) Applicants: Institut D'Investigacions Biomediques August Pi Isunyer (IDIBAPS), Barcelona (ES); Baylor College of Medicine, Houston, TX (US); Memorial Sloan Kettering Cancer Center, New York, NY (US); Universitat de Barcelona, Barcelona (ES)

(72) Inventors: Rachel Schiff, Bellaire, TX (US); Mothaffar Rimawi, Houston, TX (US); Jamunarani Veeraraghavan, Houston, TX (US); C. Kent Osborne, Houston, TX (US); Carolina Gutierrez, Houston, TX (US); Aleix Prat, Barcelona (ES); Jorge Reis-Filho, New York, NY (US)

(21) Appl. No.: 17/998,240

(22) PCT Filed: May 12, 2021
(86) PCT No.: PCT/US2021/070543

§ 371 Date: Nov. 8, 2022

Related U.S. Application Data
(60) Provisional application No. 63/023,785, filed on May 12, 2020.

(51) Int. Cl. A61K 31/517; A61K 31/506; A61K 31/4706; C07K 16/32; A61P 35/00; C12Q 1/6886; G01N 33/574

(52) U.S. Cl. CPC A61K 31/517; A61K 31/506; A61K 31/4706; C07K 16/32; A61P 35/00; C12Q 1/6886; G01N 33/57484; G01N 33/57415; C12Q 2600/158; C12Q 2600/106

(57) ABSTRACT

Embodiments of the disclosure include methods and compositions related to treating an individual for HER2+ positive cancer with an appropriate treatment based on outcome of a multiparameter classifier. The methods allow for identification of HER2+ individuals that are suitable to avoid chemotherapy, in specific embodiments. Methods of the disclosure also allow for identification of HER2+ individuals that should not avoid chemotherapy. In specific embodiments, the multiparameter classifier identifies whether there is (1) a ratio of HER2 amplification, relative to a control probe, of greater than or equal to 4.5; (2) a HER2 expression level score of 3+, as determined by immunohistochemistry, in at least 90% of breast cancer cells; (3) whether there is a HER2-enriched molecular subtype; and (4) whether the individual has a wildtype PIKC3A gene.

This application claims priority to U.S. Provisional Patent Application Ser. No. 63/023,785, filed May 12, 2020, which is incorporated by reference herein in its entirety.

This invention was made with government support under W81XWH-17-1-0579 and W81XWH-17-1-0580 awarded by the Department of Defense. The government has certain rights in the invention.

### BACKGROUND

### I. Technical Field

Embodiments of the disclosure relate at least to the fields of cell biology, molecular biology, diagnostics, cancer biology, and medicine.

### II. Background

Breast cancers have a high level of heterogeneity, making it difficult to choose the appropriate treatment for a patient. It is important to select a therapeutic approach that maximizes efficacy while minimizing toxicity. Various clinical trials have shown 25-30% pathologic complete response (pCR) rates in patients with human epidermal growth factor receptor 2 (HER2)+ breast cancer treated with lapatinib and trastuzumab (with endocrine therapy if estrogen receptor (ER)+), but no chemotherapy. There is a need for methods for identifying patients who will respond to chemotherapy-free therapy, thus limiting chemotherapy-related toxicities in these patients while maintaining optimal outcomes.

### SUMMARY

Embodiments of the disclosure encompass systems, methods, and compositions for HER2+ cancer therapy and determination thereof. Specific embodiments of the disclosure provide a molecular classifier for a targeted therapy for HER2+ cancer, including a HER2+ breast cancer. Breast cancers are well known for a high level of heterogeneity that makes it difficult to choose the appropriate treatment for an individual. Combined therapeutic approaches have been used to try to maximize treatment efficacy; however, it is useful to maximize treatment efficacy while minimizing toxicity that can occur from cancer treatments like chemotherapy. In breast cancers that rely on an increase in HER2 expression (HER2+), HER2 inhibition therapy is typically utilized in combination with chemotherapy. It is believed that HER2 inhibition may not be enough because of compensatory signaling from alternative pathways or downstream components. However, therapies for specifically HER2-dependent tumors (i.e., those lacking compensatory signaling) are more effective with an increase in HER2 inhibition therapy without chemotherapy. Up to this point, it has been difficult to identify which tumors are specifically HER2-dependent. This disclosure describes a molecular classifier to identify HER2+ breast cancers that are specifically HER2-dependent. In particular, this molecular classifier assays for very high levels of HER2 and also ascertains whether there is no activation of a downstream component of the HER2 pathway, PI3K. The inventors have developed and validated this classifier using specimens from previous clinical trials and shown that it is an excellent predictor for response to this specific treatment. By using this classifier effectively, this can identify appropriate treatment and improve patient outcomes for HER2+ breast cancer patients.

Embodiments of the disclosure encompass use of a multiparameter classifier that allows for determination of whether or not an individual with HER2+ cancer will need chemotherapy. In certain cases following determination of the outcome of the multiparameter classifier, the individual will be able to avoid chemotherapy, whereas in other cases following determination of the outcome of the multiparameter classifier the individual will need chemotherapy. Specific methods determine identification of HER2+ individuals that can be spared chemotherapy, including for breast cancer, for example. The methods apply to any HER2+ cancers including at least breast, gastric, etc.

In particular embodiments, provided herein is a multiparameter classifier to predict effectiveness of a response to one or more HER2-targeted therapies without chemotherapy, including for HER2+ breast cancer. In specific cases, provided herein is a multiparameter classifier to predict effectiveness of a response to trastuzumab alone or in combination with other anti-HER2 therapy without chemotherapy, including for HER2+ breast cancer. Specific aspects identify patients with HER2+ breast cancer who will benefit from HER2-targeted therapy alone, without chemotherapy and also identify patients with HER2+ breast cancer who will benefit from HER2-targeted therapy but who also will need chemotherapy. In at least some embodiments, for breast cancer, clinical HER2+ is defined per the American Society of Clinical Oncology (ASCO) guidelines as ≥2 HER2 FISH ratio and/or ≥10% 3+ cells by immunohistochemistry. The ASCO guidelines are known in the art and are incorporated by reference herein in their entirety (see Wolff A C, Hammond E H, Allison K H, et al. Human Epidermal Growth Factor Receptor 2 Testing in Breast Cancer: American Society of Clinical Oncology/College of American Pathologists Clinical Practice Guideline Focused Update. Arch Pathol Lab Med. 2018; 142(11):1364-1382, and also see Human Epidermal Growth Factor Receptor 2 Testing in Breast Cancer: American Society of Clinical Oncology/College of American Pathologists Clinical Practice Guideline Focused Update. Antonio C. Wolff, M. Elizabeth Hale Hammond, Kimberly H. Allison, Brittany E. Harvey, Pamela B. Mangu, John M. S. Bartlett, Michael Bilous, Ian O. Ellis, Patrick Fitzgibbons, Wedad Hanna, Robert B. Jenkins, Michael F. Press, Patricia A. Spears, Gail H. Vance, Giuseppe Viale, Lisa M. McShane, and Mitchell Dowsett. Journal of Clinical Oncology 2018 36:20, 2105-2122.) In specific cases, the molecular classifier can identify patients who may not benefit from a chemotherapy-sparing strategy and may need more therapy such as chemotherapy and/or additional targeted treatments.

In particular aspects, the multiparameter classifier of the disclosure comprises identification of HER2 gene amplification (including as expressed as a ratio of HER2 gene copies to the number of control probe copies), and protein levels, intratumoral heterogeneity (ITH), HER2-enriched (E) subtype, and PIK3CA mutation status. In specific cases, an individual is not treated with chemotherapy when it is determined from cancer samples therefrom to (a) have a ratio of HER2 gene amplification, relative to a control probe, of greater than or equal to 4.5; (b) have a HER2 expression level score of 3+, as determined by immunohistochemistry, in at least 90% of breast cancer cells; (c) to be of a HER2-enriched molecular subtype; and (d) to have a wildtype PIK3CA gene. In alternative cases, an individual is treated with chemotherapy (and in some cases other cancer therapies, including HER2-targeted therapies) when it is determined from cancer samples therefrom to (a) have a ratio of HER2 gene amplification, relative to a control probe, of less than or equal to 4.4; (b) not to have a HER2 expression level score of 3+, as determined by immunohistochemistry, in at least 90% of breast cancer cells; (c) not to be of a HER2-enriched molecular subtype; and/or (d) not to have a wildtype PIK3CA gene (such as have an activating mutation in this gene).

In some embodiments, there is a method for treating a subject for cancer comprising providing a therapeutically effective amount of a HER2-targeted therapy to the subject, wherein one or more cancer samples from the subject have been determined: (a) to have a ratio of HER2 amplification, relative to a control probe (such as CEP17), of greater than or equal to 4.5; (b) to have a HER2 expression level score of 3+, as determined by immunohistochemistry, in at least 90% of breast cancer cells; c) to be of a HER2-enriched molecular subtype; and d) to have a wildtype PIK3CA gene.

In one embodiment, there is a method for identifying a subject with breast cancer as being sensitive to a HER2-targeted therapy but not in need of chemotherapy, the method comprising detecting, from one or more breast cancer samples from the subject: (a) a ratio of HER2 amplification, relative to a control probe (such as CEP17), of greater than or equal to 4.5; (b) greater than 90% of cells as having a HER2 expression level score of 3+, as determined by immunohistochemistry; (c) a HER2-enriched molecular subtype; and (d) a wild-type PIK3CA gene.

In some cases, the HER2-targeted therapy comprises one or more HER2 small molecule inhibitors, one or more anti-HER2 antibodies or antibody-like molecules and/or one or more HER2 small molecule inhibitors. In particular cases, the method comprises achieving a pathologic complete response in a subject. In particular aspects, (a) comprises performing gene amplification on DNA from one or more breast cancer samples. Any part of any method herein may utilize chromogenic in situ hybridization (CISH), fluorescent in situ hybridization (FISH), polymerase chain reaction, and/or next generation DNA sequencing on one or more samples from the subject. In some cases, (c) comprises performing a gene expression analysis of one or more breast cancer samples; the analysis may be of a PAM50 gene signature. In some cases, the gene expression analysis is a DNA microarray or is RNA sequencing. In particular embodiments, (d) comprises sequencing DNA from one or more breast cancer samples. In specific cases the method does not comprise providing a chemotherapy to the subject.

In particular embodiments, the HER2-targeted therapy comprises one or more HER2 small molecule inhibitors and/or one or more anti-HER2 antibodies or antibody-like molecules. Examples of HER2 small molecule inhibitors include lapatinib, neratinib, tucatinib, afatinib, or a combination thereof. In specific cases, the anti-HER2 antibody or antibody-like molecule is trastuzumab and/or pertuzumab.

In certain embodiments, the subject has stage I, II, III, IIIa, or IV breast cancer. In certain aspects, the subject has estrogen receptor (ER)-positive breast cancer and wherein the method further comprises providing a hormone therapy to the subject. In specific cases, the subject has ER-negative breast cancer.

In specific embodiments, the HER2-targeted therapy comprises one or more HER2 small molecule inhibitors.

In particular cases, the subject is identified as being sensitive to the HER2-targeted therapy in the absence of chemotherapy. In certain embodiments of the method, the method further comprises providing to the subject a cancer therapy, wherein the cancer therapy comprises the HER2-targeted therapy. In some cases, the cancer therapy does not comprise a chemotherapy. In one embodiment, there is a method for treating a subject for breast cancer comprising providing a therapeutically effective amount of a cancer therapy to the subject, the method comprising providing to the subject a HER2-targeted therapy, wherein: (a) the method does not comprise providing a chemotherapy to the subject if one or more breast cancer samples from the subject have been determined: (i) to have a ratio of HER2 amplification, relative to a control probe, of greater than or equal to 4.5; (ii) to have a HER2 expression level score of 3+, as determined by immunohistochemistry, in at least 90% of breast cancer cells; (iii) to be of a HER2-enriched molecular subtype; and (iv) to have a wildtype PIK3CA gene; or (b) the method further comprises providing a therapeutically effective amount of a chemotherapy and/or one or more other therapies to the subject if one or more breast cancer samples from the subject have been determined: (i) to have a ratio of HER2 amplification, relative to a control probe, of less than 4.5; (ii) to have a HER2 expression level score of 3+, as determined by immunohistochemistry, in less than 90% of breast cancer cells; (iii) to not be of a HER2-enriched molecular subtype; or (iv) to have a mutant PIK3CA gene.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a decision tree corresponding to the molecular classifier of the present disclosure: GPA ratio ≥4.5 AND +3%≥90 AND HER2-enriched AND phosphatidylinositol-4,5-bisphosphate 3-kinase catalytic subunit alpha (PIK3CA) wild-type. Shown are numbers of patients from the TBCRC023 clinical trial achieving a pathological complete response (pCR) and those not achieving a pCR (non-pCR) based on each classification.

FIGS. 2A and 2B show the HER2 gene ratio and HER2% 3+ cells (classified by immunohistochemistry) for patients achieving a pCR (open circles) and those not achieving a pCR (closed circles) from the TBCRC023 (FIG. 2A) and PAMELA (FIG. 2B) clinical trials.

FIG. 3 shows the HER2 ratio, HER2-E status, and PIK3CA mutation status (mut vs. NonMut) patients from the TBCRC023 clinical trial. Patients achieving a pCR are shown in black, with patients not achieving a pCR shown in red.

FIGS. 4A and 4B show examples of oncogenic signaling pathways whose members may be genetically analyzed as part of methods of the disclosure (reproduced from Sanchez-Vega et al., Oncogenic Signaling Pathways in The Cancer Genome Atlas, Cell 173, 321-337, 2018).

### DETAILED DESCRIPTION

### I. Examples of Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “chemotherapy” as used herein refers to cytotoxic substances that do not target a specific gene product, such as HER2.

The term “HER2-enriched (E) subtype” as used herein refers to the tumor subtype characterized by higher expression of HER2, HER2-amplicon genes (e.g., GRB7), and receptor tyrosine kinases including FGFR4 and EGFR, and lower expression of luminal-related genes compared to the luminal subtypes. HER2-E tumors are likely to have the highest activation of EGFR and/or HER2 pathway and the ones to benefit the most from dual HER2 blockade. In specific embodiments, the test utilizes assessment of 50 genes, for example.

The term “HER2-targeted therapy” as used herein refers to treatment for a HER2+ cancer cell that is dependent upon binding to HER2, blocking HER2 signaling, or inhibiting HER2 activity. The HER2-targeted therapy may be of any kind of molecule, including protein (such as an antibody, including a monoclonal antibody) or small molecule, for example. In some cases, HER2-targeted therapy may also bind to or target other members of the HER family.

Several trials have shown 25-30% pathologic complete response (pCR) rates in patients with HER2+ breast cancer treated with dual HER2-targeted therapy plus endocrine therapy if the tumors co-express estrogen receptor (ER+), but no chemotherapy. The challenge, however, is to develop a clinical assay that can identify this subset of individuals who may benefit from this chemotherapy-sparing de-escalated treatment approach. Using methods described herein, there is provided a multi-parameter molecular classifier with the following components and cutoffs: (1) HER2 ratio (HER2:CEP17 ratio by Chromogenic in situ hybridization (CISH) or fluorescent in situ hybridization (FISH))≥4.5; and (2) percent (%) 3+ (by immunohistochemistry)≥90%; and (3) PIK3CA-wild type (by genomic analyses, for example); and (4) HER2-enriched subtype (by PAM50 subtyping). This disclosure encompasses a molecular classifier for translating and implementing chemotherapy-sparing treatment strategy for suitable individuals.

### II. Multiparameter Classifier and Use Thereof

Embodiments of the disclosure encompass a multiparameter classifier and its use to determine a suitable therapy for a HER2+ individual. The multiparameter classifier methods utilized herein may be employed by the same entity or at generally the same step as determining whether or not an individual has a HER2+ cancer. For example, an individual suspected of having a cancer or known to have a cancer may be determined to be HER2+ in the same assay or assays as those including use of the present multiparameter classifier. The same sample from an individual suspected of having or known to have cancer may be used to identify whether or not the individual is HER2+ and also be used for the multiparameter classifier. In other cases, different samples from an individual suspected of having or known to have cancer may be used to identify whether or not the individual is HER2+ and also be used for the multiparameter classifier. The HER2+ cancer may be of any stage and may be newly diagnosed untreated stage IV (metastatic), in some cases. In specific embodiments, the HER2+ cancer is a primary cancer, and including a cancer that has not yet been treated.

In particular embodiments, the multiparameter classifier employs four different parameters to ascertain a particular therapy for a HER2+ individual. Any methods herein may comprise, consist of, or consist essentially of four different parameters to ascertain a particular therapy for a HER2+ individual. In particular embodiments, the methods determine a suitable therapy for an individual because that individual is not subject to an unnecessary treatment for a HER2+ cancer, including at least one that is cumulative or duplicative to another therapy the individual will receive and/or is receiving and/or that would impart deleterious side effects for the individual. In specific cases, a therapy is avoided because the cancer would have been treated by another therapy or therapies that the individual is already receiving and/or will receive. In specific cases, a therapy is avoided whose mechanism of action is not specific to targeting HER2 in the surface of cancer cells in the individual.

In particular embodiments, use of a multiparameter classifiers identifies an individual as suitable to avoid a chemotherapy if the individual has (a) a ratio of HER2 amplification identified as a ratio of the number of HER2 gene copies relative to the number of copies of a control probe, of greater than or equal to 4.5; and (b) a HER2 expression level score of 3+, as determined by immunohistochemistry, in at least 90% of breast cancer cells; and (c) has a HER2-enriched molecular subtype; and (d) has a wildtype (no activating mutations or amplifications) PIK3CA gene.

One parameter of the multiparameter classifier concerns determining HER2 gene copies in comparison to a control probe. In specific embodiments, the parameter concerns the number of HER2 gene copies divided by the number of chromosome 17 copies as determined by a control probe that recognizes the centromere or some other gene on chromosome 17. This parameter shows whether the HER2 gene has been amplified (more copies of that gene) above other genes on the chromosome. When the ratio of HER2 copy number, relative to a control probe, is greater than or equal to 4.5, this information may be utilized with the other parameters to determine a suitable therapy (or omission of a therapy) for HER2+ cancer in an individual. The determination of HER2 amplification levels may be performed by analysis of HER2 copy number (such as >10). Examples of assays to determine HER2 and control probe levels or to determine HER2 protein levels includes in situ hybridization (including fluorescent in situ hybridization or colorimetric in situ hybridization), immunohistochemistry, gene protein assay (that allows the simultaneous analysis by immunohistochemistry of HER2 protein, HER2 gene amplification levels with in situ hybridization, and intra-tumor heterogeneity that may or may not be performed on a single slide), and next-generation DNA sequencing, and so forth. An example of a control probe to utilize for comparison in a ratio with HER2 includes chromosome enumeration probe 17 (CEP17). (In other methods, such as genomic copy number PCR (see, for example, commercially available assays such as from Qiagen (Germantown, Md.). One can utilize housekeeping genes for qRT-PCR for RNA expression analysis or in proteomic assays like western blot for normalization; examples include glyceraldehyde 3-phosphate dehydrogenase (GAPDH), beta glucuronidase (GUSB), beta-2-microglobulin (B2M), and so forth. In particular embodiments, the ratio of HER2 expression relative to a control probe is greater than or equal to (or greater than or equal to about) 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, 10, 11, 12, 13, 14, 15, 20, 25, 30, or greater, as examples.

One parameter of the multiparameter classifier concerns determining a HER2 expression level score based on complete membrane staining by IHC. In the present multiparameter classifier, there must be a score of 3+, as determined by immunohistochemistry, in at least 90% of breast cancer cells. In specific embodiments, the score is determined to be such in at least (or at least about) 90, 91, 92, 93, 94, 95, 96, 97, 98, 99, or 100% of breast cancer cells with strong and complete staining of the cell membrane and not the 2+ defined as weak to moderate complete staining of cell membrane or 1+ defined as faint incomplete staining of cell membrane.

One parameter of the multiparameter classifier comprises determining whether or not the individual has a HER2-enriched (E) subtype, such as by performing PAM50 subtyping or research-based PAM50, as examples. Determination of an enriched subtype may use a qualitative assay that utilizes gene expression data, and it may be weighted together with clinical variables to generate a risk category and numerical score. For example, as with the tumor profiling test PAM50 (defined by differential expression of 50 genes) (Prosigna®), the gene expression profile of an individual's tumor is compared with prototypical molecular profiles to determine the degree of similarity; for the PAM50 test, the gene expression profile of an individual's tumor is compared with each of the 4 PAM50 prototypical molecular profiles to determine the degree of similarity (Wallden et al., 2015, BMC Med Genomics. 2015; 8: 54).

One parameter of the multiparameter classifier concerns determining the PI3K pathway status, such as determining whether there are genomic alterations in PIK3CA (also known as CLAPO, CLOVE, CWS5, MCAP, MCM, MCMTC, PI3K, PI3K-alpha, or p110-alpha) or any member of the PI3K pathway. In specific embodiments, the PI3K pathway activity may also be impacted because of one or multiple genomic aberrations (including but not limited to copy number alterations, single nucleotide mutations, INDELs, and somatic rearrangements also known as fusion genes) in members of the PI3K pathway itself or of those in the upstream or downstream signaling, including but not limited to the Receptor Tyrosine Kinase (RTK)/Ras pathway, for example. Activation of the RTKs/Ras, etc, not only activates the PI3K pathway, but provides additional/alternative proliferative and survival signaling to reduce “HER2 addiction”. Therefore, in some cases in addition to assaying for wild-type status of PIK3CA, one may also determine whether or not there is activation of RTKs/Ras or related signaling pathways that would cause a similar output. In specific embodiments, it is determined whether or not an individual has wild type PIK3CA and/or high PTEN levels in cancer cells of the individual. In certain cases, the PIK3CA mutation status identifies individuals whose tumors are “addicted” to HER2 signaling and are likely to achieve pathologic complete response (pCR) from a chemotherapy (CTX)-sparing de-escalation strategy. Such determinations may include DNA sequencing, for example. As used herein, wild-type PIK3CA includes no mutations in the PIK3CA DNA or protein sequence, although it may also include single nucleotide polymorphisms, for example, that do not substantially affect the activity or expression of the gene product. In at least some cases, mutations in PIK3CA would include genomic aberration at the gene level, i.e., gene amplification or rearrangements/fusion genes. As one example to compare the PIK3CA nucleotide sequence to, one may refer to National Center for Biotechnology Information's GenBank® Accession No. NM_006218, which is incorporated herein by reference. As one example to compare the PIK3CA protein sequence to, one may refer to GenBank® Accession No. NP_006209, which is incorporated herein by reference. Determination of whether or not the PIK3CA is wild-type may be performed by DNA sequencing of any kind, including by Memorial Sloan Kettering-Integrated Mutation Profiling of Actionable Cancer Targets (MSK-IMPACT), a hybridization capture-based next-generation sequencing assay for targeted deep sequencing, for example. Examples of mutations in PIK3CA include those within the helical domain (exon 9, commonly E542 and E545) and/or the kinase domain (exon 20, commonly H1047), including H1047R and E545K, as specific examples.

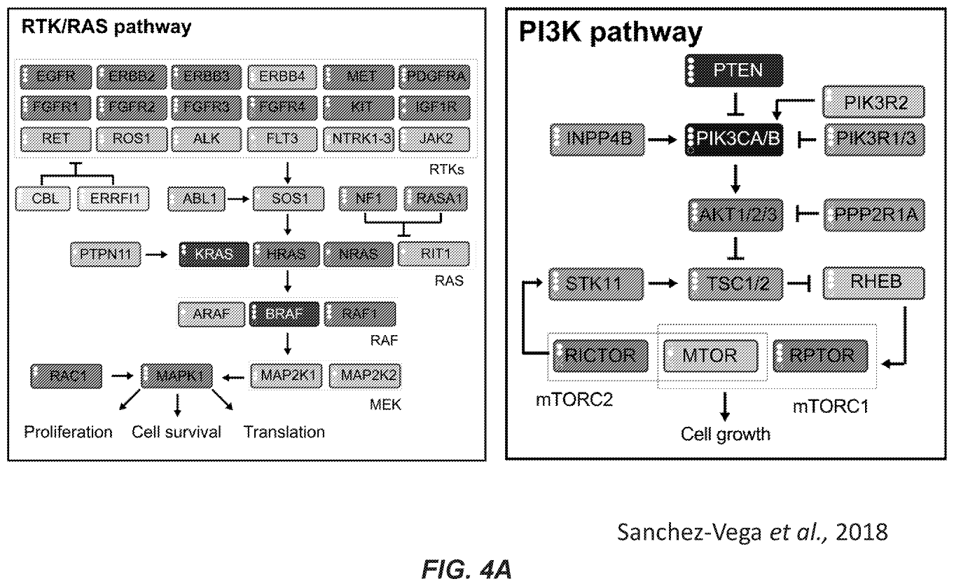

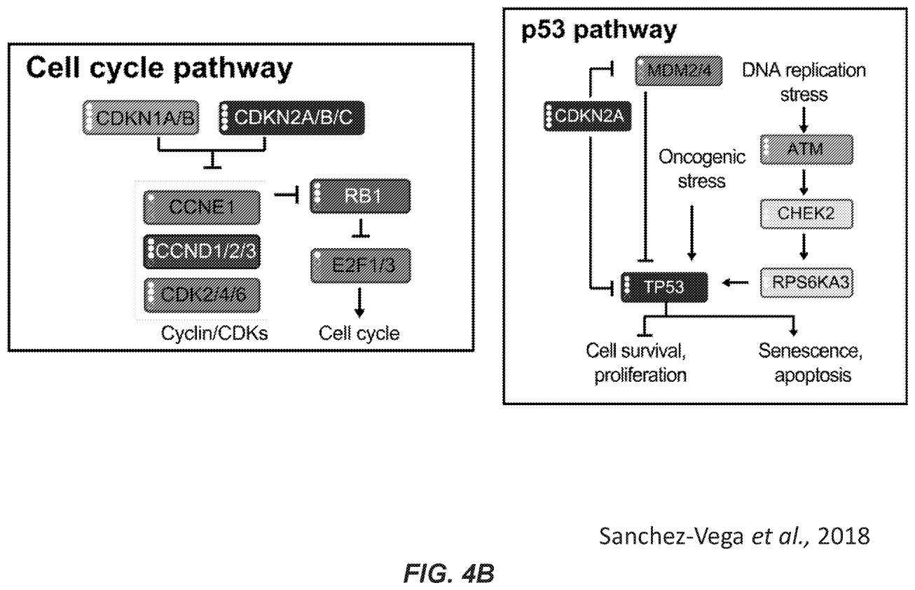

In particular embodiments, the above-mentioned and other genetic parameters may be determined as part of the aforementioned four-part multiparameter classifier or as additional parameters to the aforementioned four-part multiparameter classifier, and these other genetic parameters may be a part of genetic alterations in a variety of signaling pathways associated with HER2 directly or indirectly. See Sanchez-Vega et al., Oncogenic Signaling Pathways in The Cancer Genome Atlas, Cell 173, 321-337 (2018), incorporated by reference herein in its entirety. For example, activating mutations in HER2, also known to be associated with resistance, may jeopardize the outcome of HER2-targeted therapy, without chemotherapy. Activating mutations or amplifications of other HER family members (HER1, HERS, or HER 4) or increased levels of the mRNA and/or protein of the HER receptors and their ligands (including any member of the related RTK pathway) may also have an impact on outcome and may be analyzed as part of methods of the disclosure. In some embodiments, genomic alterations in members of the P53 and/or RB pathway, which result in pathway inactivation, may also impact outcome to HER2-targeted therapy and may be ascertained as part of methods of the disclosure. For examples of oncogenic signaling pathway members whose mutation or activation or amplification may be determined, see FIGS. 4A and 4B, reproduced from Sanchez-Vega et al., Oncogenic Signaling Pathways in The Cancer Genome Atlas, Cell 173, 321-337, 2018).

Use of the multiparameter classifier of the disclosure provides information to a medical provider to determine a suitable therapy including, at least in some cases, whether or not chemotherapy may be avoided by the individual.

Examples of chemotherapy avoided by certain methods of the disclosure (and that are not HER2-targeting or HER2-specific) include at least certain alkylating agents, plant alkaloids, antitumor antibiotics, antimetabolites, topoisomerase inhibitors, and combinations thereof. In specific embodiments, chemotherapy that may be avoided includes anthracyclines, such as doxorubicin, pegylated liposomal doxorubicin, and epirubicin; taxanes, such as paclitaxel and docetaxel; 5-fluorouracil or capecitabine; cyclophosphamide; platinum agents, such as carboplatin; Vinorelbine; Capecitabine; Gemcitabine; Ixabepilone; Eribulin, and combinations thereof.

In certain embodiments, when an individual in need of cancer treatment has one or more cancer samples that have been determined:

(a) to have the level of HER2 amplification, determined by the ratio of HER2 copy number relative to a control probe of greater than or equal to 4.5 in HER2+ breast or other HER2+ cancer cells and/or it may be determined by the absolute HER2 copy number of ≥10 in HER2+ breast or other HER2+ cancer cells;

(b) to have a HER2 expression level score of 3+, as determined by immunohistochemistry, in at least 90% of cancer cells;

(c) to be of a HER2-enriched molecular subtype; and

(d) to have a wildtype PIK3CA gene, the individual is provided an effective amount of one or more therapies for the cancer that are not chemotherapy, and in particular embodiments the one or more therapies are HER2-targeted therapies.

In particular embodiments, there are methods for treating a subject for cancer comprising providing a HER2-targeted therapy to the subject, wherein one or more cancer samples from the subject have been determined:

(a) to have a ratio of HER2 amplification, relative to a control probe (and including HER2 amplification expressed as a ratio of the number of HER2 gene copies to the number of control gene probe copies), of greater than or equal to 4.5, and/or it may be determined by the absolute HER2 copy number of ≥10;

(b) to have a HER2 expression level score of 3+, as determined by immunohistochemistry, in at least 90% of cancer cells;

(c) to be of a HER2-enriched molecular subtype; and

(d) to have a wildtype PIK3CA gene, wherein the subject is not provided one or more chemotherapies.

In particular embodiments, there are methods for reducing a tumor load for a subject comprising providing a HER2-targeted therapy to the subject, wherein one or more HER2-positive cancer samples from the subject have been determined:

(a) to have a ratio of HER2 expression, relative to a control probe (and including HER2 amplification expressed as a ratio of the number of HER2 gene copies to the number of control gene probe copies), of greater than or equal to 4.5 and/or it may be determined by the absolute HER2 copy number of ≥10;

(b) to have a HER2 expression level score of 3+, as determined by immunohistochemistry, in at least 90% of cancer cells;

(c) to be of a HER2-enriched molecular subtype; and

(d) to have a wildtype PIK3CA gene, wherein the subject is not provided one or more chemotherapies.

In certain embodiments, a subject is provided a therapeutically effective amount of a chemotherapy to the subject if one or more cancer samples from the subject have been determined:

(i) to have a ratio of HER2 expression, relative to a control probe (and including HER2 amplification expressed as a ratio of the number of HER2 gene copies to the number of control gene probe copies), of less than 4.5 and/or it may be determined by the absolute HER2 copy number of less than 10;

(ii) to have a HER2 expression level score of 3+, as determined by immunohistochemistry, in less than 90% of cancer cells;

(iii) to not be of a HER2-enriched molecular subtype; or

(iv) to have a mutant PIK3CA gene. The chemotherapy may be administered with HER2-targeted therapy if the tumor is clinically HER2+ by current ASCO guidelines, i.e., HER2 ratio ≥2 or %3+≥10%, and/or additional targeted treatments.

### III. Administration of Therapeutic Compositions

In particular embodiments, an individual in need thereof is provided one or more therapeutic compositions. The therapeutic composition(s) may be determined based on analysis of the multiparameter classifier described herein. In specific embodiments, an effective amount of one or more cancer therapies are provided to an individual following determination of specific parameters for the multiparameter classifier of the disclosure, and the cancer may be of any kind of HER2+ cancer. In some cases, the therapeutic composition(s) will not comprise chemotherapy (following determination of the multiparameter classifier outcome), whereas in other cases the therapeutic compositions may comprise chemotherapy (following determination of the multiparameter classifier outcome).

Any therapy provided herein may comprise administration of one or more HER2-targeted therapies, including antibodies or the like and/or small molecule(s). In specific examples, a HER2-targeted therapy that is an antibody is given to an individual in addition to a HER2-targeted therapy that is a small molecule, and the individual may or may not also be given chemotherapy, determined upon the multiparameter classifier outcome. In specific cases, the individual is given lapatinib and trastuzumab. Any combination therapy using two or more HER2-targeted therapies (for example, either two antibodies such as trastuzumab and pertuzumab or an antibody plus small molecule HER2 inhibitor, such as lapatinib and trastuzumab) may be administered in any suitable manner known in the art, including sequentially and/or concurrently. The two or more HER2-targeted therapies may be administered in the same composition or in different compositions.

The molecular classifier provides a predictor for HER2-targeted therapy only if the tumor meets the criteria of the molecular classifier. HER2-targeted therapy plus chemotherapy can be used for tumors not meeting the criteria of the molecular classifier. Therefore, in some cases the therapy provided herein may comprise administration of a combination of therapeutic agents, such as a first cancer therapy (e.g., a HER2-targeted therapy) and a second cancer therapy (e.g., a chemotherapy). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 mg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition(s) also depend on the judgment of the practitioner and may be tailored to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing, and such considerations are routine in the art.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Detecting HER2 Genetic Signature and Associated Molecular Characteristics

Particular embodiments concern the methods of detecting a HER2 genetic signature in an individual. In some embodiments, the method for detecting the HER2 genetic signature provides information about any of the parameters of the multiparameter classifier. In specific embodiments, it may include utilizing selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

A. Single Nucleotide Polymorphism (SNP) Detection

Although detection of a SNP in HER2 may be not utilized in certain aspects of the multiparameter classifier, in some cases distinguishing between a SNP in PIK3CA or other genes in the PI3K pathway and a mutation that affects expression and/or activity may be performed. One may employ any of the known general methods for detecting mutations or SNPs, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect a mutation or SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular mutation or SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the mutation or SNP. For example, one selective oligonucleotide probe binds to a complementary strand that has an A nucleotide at the mutation or SNP on the coding strand but not a G nucleotide at the SNP on the coding strand, while a different selective oligonucleotide probe binds to a complementary strand that has a G nucleotide at the mutation or SNP on the coding strand but not an A nucleotide at the mutation or SNP on the coding strand. Similar methods could be used to design a probe that selectively binds to the coding strand that has a C or a T nucleotide, but not both, at the mutation or SNP. Thus, any method to determine binding of one selective oligonucleotide probe over another selective oligonucleotide probe could be used to determine the nucleotide present at the SNP.

One method for detecting mutations or SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the mutation or SNP position. In some embodiments, the probe is generally designed so the 3′ end of the probe pairs with the mutation or SNP. Thus, if the probe has the correct complementary base to pair with the particular nucleotide at the mutation or SNP, the probe will be extended during the amplification step of the PCR. For example, if there is a T nucleotide at the 3′ position of the probe and there is an A nucleotide at the mutation or SNP position, the probe will bind to the respective mutation or SNP and be extended during the amplification step of the PCR. However, if the same probe is used (with a T at the 3′ end) and there is a G nucleotide at the mutation or SNP position, the probe will not fully bind and will not be extended during the amplification step of the PCR.

In some embodiments, the mutation or SNP position is not at the terminal end of the PCR primer, but rather located within the PCR primer. The PCR primer should be of sufficient length and homology in that the PCR primer can selectively bind to one variant, for example the mutation or SNP having an A nucleotide, but not bind to another variant, for example the SNP having a G nucleotide. The PCR primer may also be designed to selectively bind particularly to the mutation or SNP having a G nucleotide but not bind to a variant with an A, C, or T nucleotide. Similarly, PCR primers could be designed to bind to the mutation or SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the mutation or SNP can be determined to have the A nucleotide and not the G nucleotide.

B. Copy Number Variation Detection

Particular embodiments of the disclosure concern methods of detecting a copy number variation (CNV) of HER2, and including determination whether or not there is amplification of HER2 (including in a quantitative manner) compared to the number of control probe copies. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array, wherein the array is capable of detecting CNVs on the entire X chromosome and/or all targets of miR-362. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

C. DNA Sequencing

In some embodiments, HER2 (and/or a gene in the PI3K pathway, such as PIK3CA) DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

In at least some cases, the MSK-IMPACT platform is utilized to sequence HER2 (and/or a gene in the PI3K pathway, such as PIK3CA).

D. RNA Sequencing

In some embodiments, HER2 RNA may be analyzed by sequencing in addition to the parameters in the presently disclosed molecular classifier. In specific embodiments, RNA of a gene in the PI3K pathway, such as PIK3CA, is analyzed for mutation detection. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

E. Proteomics

In some embodiments, HER2 (and/or a gene product from the PI3K pathway, such as PIK3CA) protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### V. Kits

Certain aspects of the present disclosure also concern kits containing compositions of the disclosure or compositions to implement methods of the disclosure. In some embodiments, kits can be used to evaluate one or more of the parameters in the multiparameter classifier. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating HER2 expression or protein production, copy number intratumoral heterogeneity, PIK3CA status (or other genes listed herein), a combination thereof, and so forth. In some embodiments, one or more cancer therapies are provided in the kit, including HER2-targeted therapy or therapies and/or chemotherapy or chemotherapies.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to HER2 and/or PIK3CA, which includes nucleic acid primers/primer sets and probes that are identical to or complementary to all or part of a gene, which may include noncoding sequences of the gene, as well as coding sequences of the gene.

In certain aspects, negative and/or positive control nucleic acids, probes, and inhibitors are included in some kit embodiments.

It is contemplated that any method or composition described herein can be implemented with respect to any other method or composition described herein and that different embodiments may be combined. The claims originally filed are contemplated to cover claims that are multiply dependent on any filed claim or combination of filed claims.

Any embodiment of the disclosure involving HER2 or PIK3CA is contemplated also to cover embodiments involving corresponding sequences that are at least 80, 81, 82, 83, 84, 85, 86, 87, 88, 89, 90, 91, 92, 93, 94, 95, 96, 97, 98, 99% identical to the sequence of the corresponding HER2 or PIK3CA nucleic acid.

### VI. Examples

The following examples are included to demonstrate embodiments of the disclosure. It should be appreciated by those of skill in the art that the techniques disclosed in the examples which follow represent techniques discovered by the inventor to function well in the practice of the methods and compositions of the disclosure. However, those of skill in the art should, in light of the present disclosure, appreciate that many changes can be made in the specific embodiments which are disclosed and still obtain a like or similar result without departing from the spirit and scope of the disclosure.

### Example 1—Identification of a Multiparameter Classifier to Predict Response to HER2 Targeted Therapy without Chemotherapy

### Overview

In this example, the inventors considered that a multiparameter classifier, comprised of HER2 gene and protein levels, intratumoral heterogeneity (ITH), HER2-enriched (E) subtype, and PIK3CA mutation status can identify patients whose tumors are “addicted” to HER2 signaling and are likely to achieve pathologic complete remission (pCR) from a chemotherapy (CTX)-sparing de-escalation strategy.

### Methods

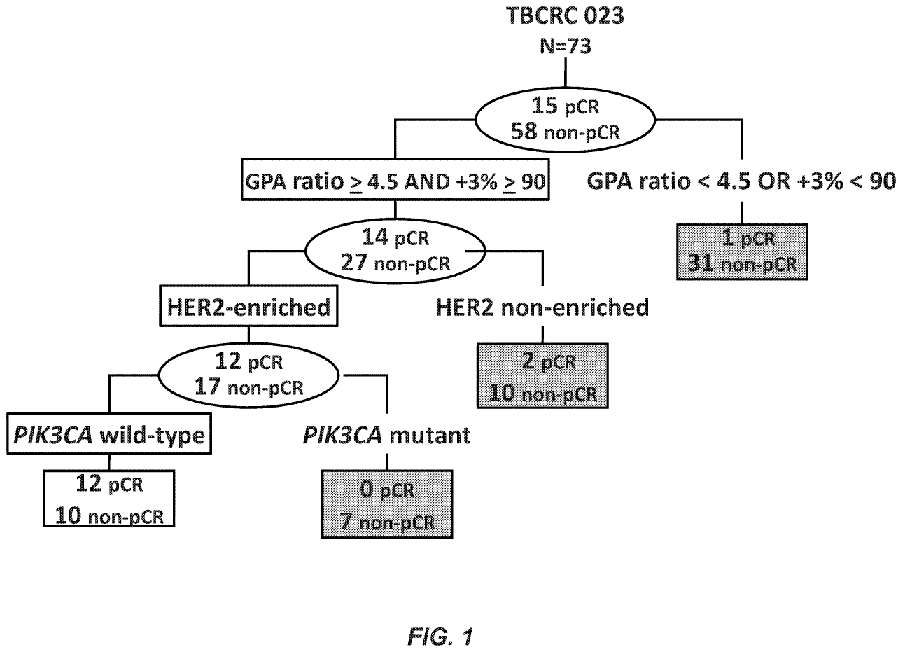

Baseline specimens from 2 clinical trials (TBCRC023 [NCT00999804] and PAMELA [NCT01973660]) of neoadjuvant chemotherapy (CTX)-sparing lapatinib (LT) (+endocrine therapy if ER+) in HER2+ breast cancer were used. HER2 protein and ITH (scored for % 3+ by immunohistochemistry), and gene amplification (HER2:CEP17 ratio and copy number (CN) by chromogenic in situ hybridization) were measured on the same slide by the dual gene protein assay (GPA). HER2-E and PIK3CA mutation status were assessed by research-based PAM50 and MSK-IMPACT platforms, respectively. A decision tree algorithm (FIG. 1) was used to determine the GPA cutoffs and to construct the classifier of response by pathologic complete response (pCR) in TBCRC023, which was then validated in PAMELA.

### Results

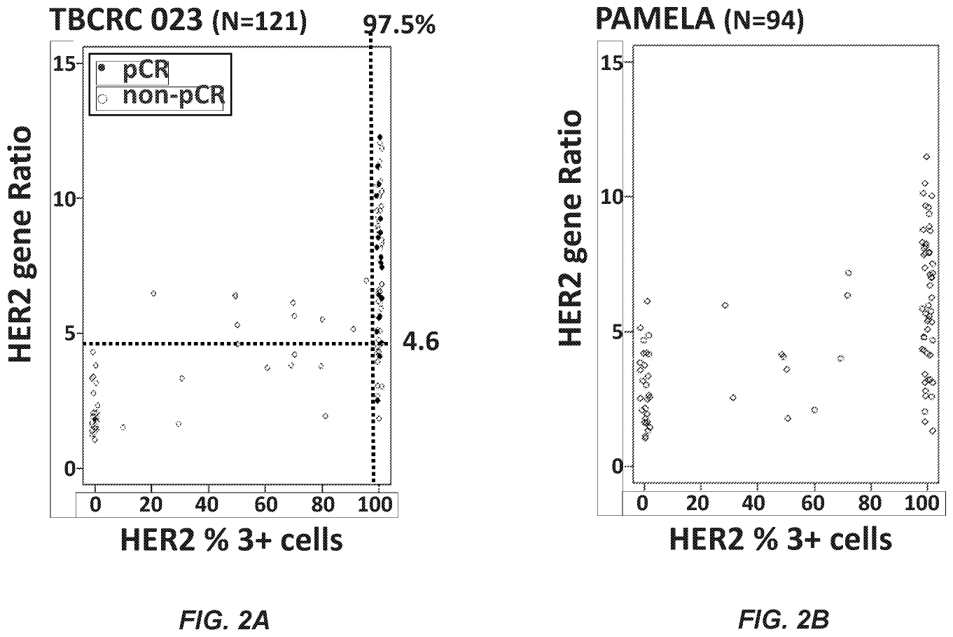

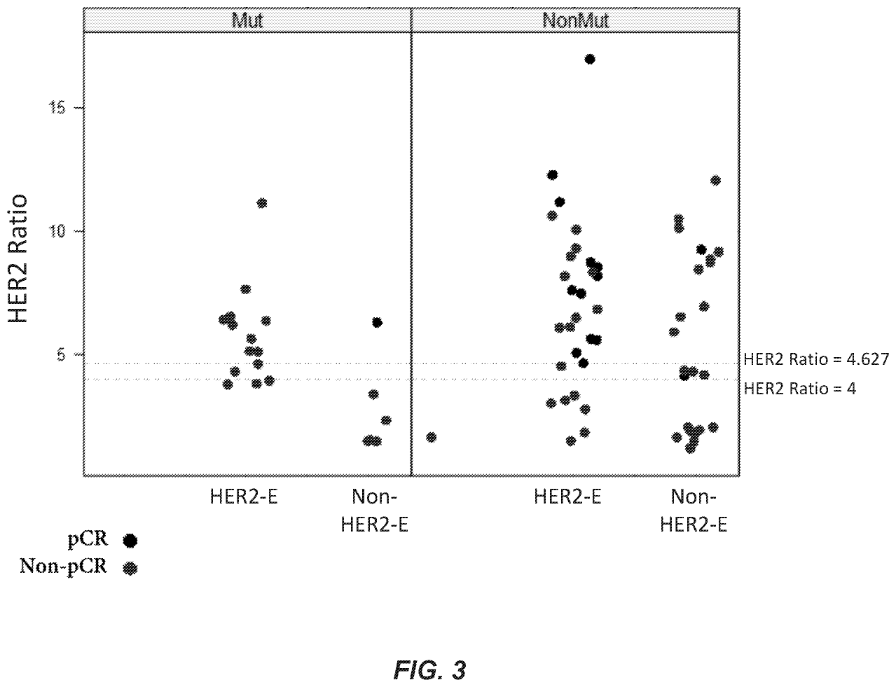

Of the evaluable patients from TBCRC023 (N=130) and PAMELA (N=151), GPA data were available for 121 and 94 cases, respectively. Both cohorts exhibited similar distributions for HER2 ratio, CN, and % 3+, and a strong correlation between HER2 ratio and CN (R>0.92). In TBCRC023, 73 cases had data from GPA, PAM50, and IMPACT, of which 15 had pCR. Recursive partitioning identified cutoffs of HER2 ratio ≥4.6 and % 3+≥97.5% in both the GPA data cohort (N=121), shown in FIG. 2A, and complete data cohort (N=73). With PAM50 and IMPACT data, the model added HER2-E and PIK3CA wild-type (wt) (FIG. 3).

For practical reasons, the classifier was locked as HER2 ratio ≥4.5 AND % 3+≥90% AND PIK3CA-wt AND HER2-E, which yielded a positive predictive value (PPV) of 55% and a negative predictive value (NPV) of 94%. Validation in PAMELA using 45 cases with data for all 3 assays yielded PPV of 44% and NPV of 82%. FIG. 2B shows the HER2 ratio and HER2% 3+ from the PAMELA cohort. These data are summarized in Table 1 below.

TABLE 1

| | | Non- | | | | |
|---|---|---|---|---|---|---|
| Trial | pCR | pCR | Sensitivity | Specificity | PPV | NPV |
| TBCRC023 | N | N | 80% | 83% | 55% | 94% |
| Predict pCR | 12 | 10 | | | | |
| Predict non-pCR | 3 | 48 | | | | |
| PAMELA | N | N | 62% | 69% | 44% | 82% |
| Predict pCR | 8 | 10 | | | | |
| Predict non-pCR | 5 | 22 | | | | |

The performance of the classifier was further evaluated using 29 cases in the TBCRC023 study and validated using 66 cases in the PAMELA study, which had partial data based on which the cases could be predicted as non-pCR. In this analysis, the classifier correctly identified 26 of the 29 non-pCRs in the TBCRC023 cohort, with a negative predictive value of 89%, and 54 of the 66 non-pCRs in the PAMELA cohort, with a negative predictive value of 81%. The results are shown in Table 2 below.

TABLE 2

| | pCR | Non-pCR | NPV |
|---|---|---|---|
| TBCRC023 | N | N | 89% |
| Predict pCR | 3 | 26 | |
| Predict non-pCR | N | N | 81% |
| PAMELA | 12 | 54 | |

The inventors have constructed a multiparameter classifier that can predict pCR with targeted therapy alone that compares to pCR rates of CTX+dual anti-HER2 therapy in unselected patients.

### Example 2—Treatment of Breast Cancer Patients Based on a Multiparameter Classifier

Breast cancer patients having HER2+ breast cancer are selected for a clinical trial and molecular analysis performed, as outlined in Example 1. Based on this analysis, the patients are segmented into two groups. A first group contains patients positive for all four of the following conditions: HER2 ratio ≥4.5, IHC % 3+≥90%, PIK3CA-wt, and HER2-E. The first group is treated with lapatinib and trastuzumab without chemotherapy. A second group contains patients who are each negative for at least one of the conditions. The second group is treated with lapatinib and trastuzumab with chemotherapy. The percentage of patients having a pathological complete response (pCR) after treatment is approximately equal between the two groups.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for treating a subject for cancer comprising providing a therapeutically effective amount of a HER2-targeted therapy to the subject, wherein one or more cancer samples from the subject have been determined:
(a1) to have a ratio of HER2 amplification, relative to a control probe, of greater than or equal to 4.5 in HER2-positive cancer cells; and/or
(a2) to have an absolute HER2 copy number of ≥10 in HER2-positive cancer cells; and
(b) to have a HER2 expression level score of 3+, as determined by immunohistochemistry, in at least 90% of HER2-positive cancer cells;
(c) to be of a HER2-enriched molecular subtype; and
(d) to have a wildtype PIK3CA gene.

2. The method of claim 1, wherein the HER2-targeted therapy comprises one or more HER2 small molecule inhibitors.

3. The method of claim 1, wherein the HER2-targeted therapy comprises one or more anti-HER2 antibodies or antibody-like molecules and/or one or more HER2 small molecule inhibitors.

4. The method of any of claims 1-3, wherein the method comprises achieving a pathologic complete response in a subject.

5. The method of any of claims 1-4, wherein the control probe is CEP17.

6. The method of any of claims 1-5, wherein (a) comprises performing gene amplification on DNA from one or more breast cancer samples.

7. The method of claim 6, wherein the method comprises performing chromogenic in situ hybridization (CISH), fluorescent in situ hybridization (FISH), polymerase chain reaction, and/or next generation DNA sequencing on one or more samples from the subject.

8. The method of any of claims 1-7, wherein (c) comprises performing a gene expression analysis of one or more breast cancer samples.

9. The method of claim 8, wherein the gene expression analysis comprises analysis of a PAM50 gene signature.

10. The method of claim 8 or 9, wherein the gene expression analysis is a DNA microarray.

11. The method of claim 8 or 9, wherein the gene expression analysis is RNA sequencing.

12. The method of any of claims 1-11, wherein (d) comprises sequencing DNA from one or more breast cancer samples.

13. The method of any of claims 1-12, wherein the HER2-targeted therapy comprises one or more HER2 small molecule inhibitors and/or one or more anti-HER2 antibodies or antibody-like molecules.

14. The method of claim 13, wherein the HER2 small molecule inhibitor is lapatinib.

15. The method of claim 13, wherein the HER2 small molecule inhibitor is neratinib.

16. The method of claim 13, wherein the HER2 small molecule inhibitor is tucatinib.

17. The method of claim 13, wherein the HER2 small molecule inhibitor is afatinib.

18. The method of claim 13, wherein the anti-HER2 antibody or antibody-like molecule is trastuzumab and/or pertuzumab.

19. The method of any of claims 1-18, wherein the method does not comprise providing a chemotherapy to the subject.

20. The method of any of claims 1-19, wherein the subject has stage I breast cancer.

21. The method of any of claims 1-19, wherein the subject has stage II breast cancer.

22. The method of any of claims 1-19, wherein the subject has stage III breast cancer.

23. The method of any of claims 1-19, wherein the subject has stage Ma breast cancer.

24. The method of any of claims 1-19, wherein the subject has stage IV breast cancer.

25. The method of any of claims 1-24, wherein the subject has estrogen receptor (ER)-positive breast cancer and wherein the method further comprises providing a hormone therapy to the subject.

26. The method of any one of claims 1-24, wherein the subject has ER-negative breast cancer.

27. A method for identifying a subject with HER2-positive cancer as being sensitive to a HER2-targeted therapy but not in need of chemotherapy, the method comprising detecting, from one or more cancer samples from the subject:
(a1) a ratio of HER2 amplification, relative to a control probe, of greater than or equal to 4.5 in HER2-positive cancer cells; and/or
(a2) to have an absolute HER2 copy number of ≥10 in HER2-positive cancer cells; and
(b) greater than 90% of cells as having a HER2 expression level score of 3+, as determined by immunohistochemistry;
(c) a HER2-enriched molecular subtype; and
(d) a wild-type PIK3CA gene.

28. The method of claim 27, wherein the HER2-targeted therapy comprises one or more HER2 small molecule inhibitors.

29. The method of claim 27, wherein the HER2-targeted therapy comprises one or more an anti-HER2 antibodies or antibody-like molecule.

30. The method of any of claims 27-29, wherein the control probe is CEP17.

31. The method of any of claims 27-30, wherein (a) comprises performing gene amplification on DNA from the one or more breast cancer samples.

32. The method of any of claims 27-31, wherein (c) comprises performing a gene expression analysis of the one or more breast cancer samples.

33. The method of claim 32, wherein the gene expression analysis comprises analysis of a PAM50 gene signature.

34. The method of claim 32 or 33, wherein the gene expression analysis is a DNA microarray.

35. The method of claim 32 or 33, wherein the gene expression analysis is RNA sequencing.

36. The method of any of claims 27-35, wherein (d) comprises sequencing DNA and/or RNA from the one or more breast cancer samples.

37. The method of any of claims 27-36, wherein the HER2-targeted therapy comprises one or more HER2 small molecule inhibitors and/or one or more anti-HER2 antibodies or antibody-like molecules.

38. The method of claim 37, wherein the HER2 small molecule inhibitor is lapatinib.

39. The method of claim 37, wherein the HER2 small molecule inhibitor is neratinib.

40. The method of claim 37, wherein the HER2 small molecule inhibitor is tucatinib.

41. The method of claim 37, wherein the HER2 small molecule inhibitor is afatinib.

42. The method of claim 37, wherein the anti-HER2 antibody or antibody-like molecule is trastuzumab and/or pertuzumab.

43. The method of any of claims 27-42, wherein the subject is identified as being sensitive to the HER2-targeted therapy in the absence of chemotherapy.

44. The method of any of claims 27-43, wherein the subject has stage I breast cancer.

45. The method of any of claims 27-43, wherein the subject has stage II breast cancer.

46. The method of any of claims 27-43, wherein the subject has stage III breast cancer.

47. The method of any of claims 27-43, wherein the subject has stage Ma breast cancer.

48. The method of any of claims 27-43, wherein the subject has stage IV breast cancer.

49. The method of any of claims 27-48, further comprising providing to the subject a cancer therapy, wherein the cancer therapy comprises the HER2-targeted therapy.

50. The method of claim 49, wherein the cancer therapy does not comprise a chemotherapy.

51. A method for treating a subject for HER2-positive cancer comprising providing a therapeutically effective amount of a cancer therapy to the subject, the method comprising providing to the subject a HER2-targeted therapy, wherein:
(a) the method does not comprise providing a chemotherapy to the subject if one or more cancer samples from the subject have been determined:
(i) to have a ratio of HER2 amplification, relative to a control probe, of greater than or equal to 4.5 in HER2-positive cancer cells; and/or
(ii) to have an absolute HER2 copy number of ≥10 in HER2-positive cancer cells; and;
(iii) to have a HER2 expression level score of 3+, as determined by immunohistochemistry, in at least 90% of breast cancer cells;
(iv) to be of a HER2-enriched molecular subtype; and
(v) to have a wildtype PIK3CA gene; or
(b) the method further comprises providing a therapeutically effective amount of a chemotherapy and/or one or more other therapies to the subject if one or more cancer samples from the subject have been determined:
(i) to have a ratio of HER2 amplification, relative to a control probe, of less than 4.5 in the HER2-positive cancer cells; or
(ii) to have an absolute HER2 copy number of <10 in the HER2-positive cancer cells; and
(iii) to have a HER2 expression level score of 3+, as determined by immunohistochemistry, in less than 90% of breast cancer cells;
(iv) to not be of a HER2-enriched molecular subtype; or
(v) to have a mutant PIK3CA gene.
